(19) United States
(12) Patent Application Publication
(10) Pub. No.: US 2026/0315596 A1
(43) Pub. Date: Oct. 15, 2026

(54) HYBRID NANOPARTICLES AS MULTIFUNCTIONAL PLATFORM FOR BRAIN TUMOR THERAPY

(71) Applicants: UNIVERSIDADE DE COIMBRA, Coimbra (PT); UNIVERSIDADE DE AVEIRO, Aveira (PT)

(72) Inventors: Maria Manuel FELICIANO DA COSTA MENDES, Coimbra (PT); Carla Sofia PINHEIRO VITORINO, Coimbra (PT); Alberto PAIS, Coimbra (PT); João José MARTINS SIMÕES DE SOUSA, Coimbra (PT); Ana Luísa DANIEL DA SILVA, Aveiro (PT); Miguel CASTELO BRANCO, Coimbra (PT); José SERENO, Coimbra (PT); Maria De Almeida VASCONCELOS ANTÓNIO, Aveiro (PT)

(21) Appl. No.: 19/119,156

(22) PCT Filed: Oct. 9, 2023
(86) PCT No.: PCT/IB2023/060125

§ 371 Date: Apr. 8, 2025

(30) Foreign Application Priority Data
Oct. 8, 2022 (PT) 118248

(51) Int. Cl. A61K 41/00; A61K 47/69; A61P 35/00

(52) U.S. Cl. CPC A61K 41/0052; A61K 47/6929; A61P 35/00

(57) ABSTRACT

The present invention discloses a hybrid metal-lipid nanoparticle for targeted therapy comprising properties of organic and inorganic nanoparticles (NPs), combined in a single formulation to obtain NPs with unique attributes. This will allow the synergistic effect between drug delivery (also known as chemotherapy) and photothermal therapy. This strategy relies on the construction of hybrid metallic-gold nanoparticles using a dual functionality by lipid nanoparticles (as organic components and drug delivery system) in the core and gold nanorods (as inorganic component and a heat delivery system) in the shell, linked by a tumor-targeting peptide. The active targeting strategy was implemented to guarantee a specific delivery to the brain by using transferrin (targeting the blood-brain barrier) and c[RGDfK] (not only used to target GB tumor barrier but also as a linker between both inorganic and organic nanoparticles).

### TECHNICAL FIELD

The invention relates to the field of medicine and biotechnology. The present solution aims at developing targeted hybrid nanoparticles, including a combination of chemo- and photothermal therapy approaches designed to improve the treatment of brain.

This invention focuses on the construction of hybrid metal-lipid nanoparticles using celecoxib-loaded lipid nanoparticles (as organic components and drug delivery system) in the core and gold nanorods (as inorganic component and a heat delivery system) in the shell. The HNPs construction relied on the covalent linkage between ultra-small (<100 nm) nanostructured lipid carriers (usNLCs) and gold nanorods (AuNRs), occurring through the binding of the amine group in octadecylamine and the carboxyl group of aspartate residue in the cyclo(L-arginyl-glycyl-L-alpha-aspartyl-D-phenylalanyl-L-lysyl) [c(RGDfK)] peptide.

The dual functionality of the linker (c[RGDfK]) is highlighted: it serves as a bond between organic and inorganic nanoparticle moieties, making them a single nanoplatform, gathering the metal- and lipid-based nanoparticles, and at the same time, as a tumor-targeting peptide.

### BACKGROUND

Cancer remains one of the leading causes of death globally, as recurrent forms commonly appear after surgical resection thus impairing the complete remission of the tumor.

Brain tumors are a heterogeneous group of primary and metastatic neoplasms in the central nervous system (CNS), characterized by poor prognosis and patient low survival rate. They are classified by the World Health Organization (WHO) according to a grade of malignancy. That is closely related to diagnosis, varying from grade I, which is characterized by lesions with low proliferative potential and possibility of cure, to grade IV (also known as glioblastoma, GB), which is described as cytological malignant, mitotically active neoplasms that are typically associated with extensive invasion of the surrounding healthy tissue and rapid proliferation linked to disease evolution.

GB is the most frequent primary brain tumor, the most aggressive and lethal in humans.

Epidemiologically, GB is a rare tumor with a global incidence of 3.69 per 100,000 people and poor prognosis with a survival rate between 16 and 18 months after diagnosis^{1,2}. This makes it a crucial public health issue.

Current management of GB frequently consists of surgical resection, followed by radiotherapy (RT) and adjuvant chemotherapy, with temozolomide (TMZ), both treatments inducing DNA damage. Resorting to the Stupp protocol, that is, radiation therapy given as 60 Gy (2 Gy per daily fraction, five times per week, over six weeks) with concomitant administration of temozolomide (TMZ, 75 mg/m² per day for seven days per week) followed by six cycles of TMZ monotherapy (150-200 mg/m² on five days in each 28-day cycle), does not significantly improve survival rate, despite being currently the gold standard of care³. However, the diagnosis is late and usually involves a neurological exam, brain scans, and a biopsy. Other drugs, including carmustine, cisplatin, carboplatin, doxorubicin, procarbazine, carmustine, lomustine and bevacizumab have been explored for GB treatment. However, according to comparative studies involving several chemotherapeutic agents, TMZ provided the highest median survival time in patients.

The clinical failure of several therapeutic approaches in GB is partly because of its highly infiltrative nature, the presence of the blood-brain barrier and tumor barrier, multidrug resistance, limited surgical resection, critical importance of the residual glioblastoma cells and glioma stem cells that have capacity to develop a new primary tumor, making complete resection at the cellular level impossible, and being responsible for the surgery-dependent malignance⁴.

TMZ is the first-line drug for the treatment of GB, for which an improvement in the overall survival and progression-free survival with the combination therapy relative to radiotherapy alone (median OS 14.6 vs. 12.1 months; P<0.001) is reported⁵. However, TMZ requires high systemic doses to reach therapeutic levels in the brain, due to its short half-life, which are associated to a large side effect spectrum, involving damage in healthy tissues. Other factors, including resistance mechanisms, absence of specificity, and poor drug accumulation in tumors, make the conventional treatment for GB limited and with low potential for clinical use. Inter- and intra-tumor heterogeneity, deregulated signalling pathways, DNA repair pathways, the persistence of cancer stem cells sub-populations and autophagy mechanisms, have been pointed out as some of the causes associated to TMZ resistance.

Despite multiple efforts over the past decades to develop new strategies to treat GB, none of them led to a better prognostic or an enhanced quality of life, when compared to the current standard of care. New therapeutic and diagnostic strategies are emerging, including those related to chemotherapy, hyperthermia therapy, immunotherapy, gene therapy and nanomedicine. These new strategies are intended to be used in combination with the current standard of care in GB, aiming at enhancing its therapeutic effect.

Nanotechnology has become a driving force for innovation in oncology, boosting advances in GB treatment. Nanoparticle (NP)-based carriers are attractive for medical applications, due to their unique characteristics, such as smaller size and, consequently, high specific surface area, great flexibility in terms of their chemical composition, and surface functionality by adsorption or covalent linkage of proteins, drugs, and probes, among others. At the same time, NPs can improve the bioavailability of therapeutic molecules by favouring their brain uptake and their tumor-targeted delivery by taking advantage of passive and active targeting.

Although there is a large number of successful preclinical studies with NPs for GB, their translation to clinical trials has only been partially mastered.

Lipid nanoparticles (LNPs) have a special interest, because they gather advantages and minimize limitations associated to conventional colloidal systems, possessing high physical and chemical stability, easy scale-up production, use of low-cost raw materials and absence or reduction of acute or chronic toxicity, because they are composed of excipients with accepted status by the regulatory authorities (e.g., on the basis of the generally regarded as safe status, GRAS). In addition, LNPs have a solid matrix at both room and body temperatures, allowing a controlled release and chemical protection of the drug. Therefore, the therapeutic efficiency is improved, because of the modulation in release combined with a better tolerability and targeting ability of the encapsulated drug to the biophase.

Solid lipid nanoparticles (SLNs) are the first generation of solid lipid-matrix nanoparticles, with a size range between 40-1000 nm and composed by solid lipids (melting point >40° C., 0.1%-30% w/w), surfactant (0.5%-5% w/w), water and/or drug(s). The choice of the solid lipid depends on drug solubility, being a critical parameter, because it can influence the drug loading capacity, release, and the stability of SLNs. However, SLNs have some limitations, such as a low drug loading capability in the lipid matrix, due its high crystalline nature, a high amount of water (70%-90%), some stability issues as a result of polymorphic transitions, leading to expulsion of the drug during the storage period.

Nanostructured lipid carriers (NLCs), the second generation of LNPs, were developed to overcome the disadvantages of SLNs. They are produced by the blend of solid lipids and liquid lipids (oils), preferably but not limited to the ratio ranging from 70:30 to 99.9:0.1. The matrix has a lower melting point than SLNs, and keeps the solid nature, despite the presence of the liquid lipid. The incorporation of the liquid lipid leads to a less organized matrix with high imperfections. This enables a higher drug loading in comparison to SLNs (more space is available for drug incorporation) and avoids the occurrence of polymorphic transitions, resulting in an increased stability during the storage period. The success of the encapsulated drugs and their stability in formulation is dependent on their solubility in the lipid matrix. On the other hand, in the case of hydrophilic drugs, a lipid conjugation strategy is considered, taking advantage of functional groups of the drug (e.g., amine group) and conjugated with the functional group of lipids or other components of the NLCs formulation (e.g., carboxylic acid group). Despite the improvement of NLCs, both these and SLNs possess a diameter typically in the range 150-300 nm5. However, to passively cross the BBB, LNPs should be less than 100 nm in diameter.

More recently, a third generation of LNPs, named as ultra-small nanostructured lipid carriers (usNLCs), has been developed combining all the previously described advantages with a higher liquid: solid lipid ratio, and consequently, higher drug loading, and a size under 100 nm. The particle size under 100 nm assumes relevance in terms of blood-brain barrier cross ability and enhanced permeability and retention in the tumor tissue.

Plasmonic nanoparticles can generate heat using a magnetic field, a laser, microwaves, or an ultrasound generator. Incidence of energy into plasmonic nanoparticles produces a coherent oscillation of electron density with rapid decay of energy via radiative or non-radiative pathways. Depending on the metal composition, shape, and size, multiple chemical and physical processes can be triggered. In this context, plasmonic nanoparticles have been considered as promising agents for delivering thermal energy in a localized manner and without the limitation of the type of cancer to be treated.

Photothermal therapy (PTT) is a non-invasive hyperthermia method that has recently attracted interest in clinical oncology. PTT converts light into heat by irradiating the tumor with an external light source, usually a near-infrared (NIR) laser. NIR light (700-1400 nm) is used because of its lower tissue absorption and scattering, which allows greater depth of penetration into the tissue. The NIR range can be divided into the first near-infrared (750-1000 nm, NIR-I) with less tissue penetration depth and the second near-infrared (1000-1400 nm, NIR-II) with a deeper tissue penetration. After irradiating PTT materials, three main processes can occur: absorption, scattering, and reflection. The hyperthermia effect is dependent on absorption. The recently developed NIR-absorbed photothermal nanoparticles can be classified into inorganic, organic, and inorganic-organic compositions. The inorganic nanoparticles have strong NIR absorption capacity and tunable surface plasmon resonance (SPR) properties, also claiming easiness of preparation, surface modification ability, and photostability.

Gold nanoparticles (AuNPs) are a suitable choice for photoabsorbers due to their tunable optical properties. AuNPs are mainly used in biological applications and can be tuned in the NIR region (750-1400 nm), also known as the biological window, where light penetration through the tissue is maximal. AuNPs have a large light scattering capability that allows multiple modalities to be applied simultaneously, including hyperthermia and imaging. Depending on size and shape, AuNPs are able to perform, simultaneously, several therapeutic and diagnostic functions, including photothermal therapy (PTT), photodynamic therapy (PDT), and imaging, respectively.

Gold nanorods (AuNRs) have a rod shape, which promotes excellent plasmonic and photothermal activity. Some characteristics of AuNRs are tunable absorption band, high stability and biocompatibility, making them suitable for injection in vivo without any systemic or organ toxicity, imaging capacity, and easy surface modification (incorporation of target molecules including drugs, nucleic acids, proteins for specific-receptor delivery). It should be recalled that plasmonic nanomaterials, such as AuNRs, are able to perform several therapeutic modalities, including PTT and/or PDT, and in combination with chemotherapeutics may cooperatively suppress cancer cells, developing synergistic effect and reversal of drug resistance. AuNRs can be seen as potential NPs for several applications such as delivery of biologically and/or chemically active molecules, imaging detection of diseases at an early stage, and tracking tumor cell proliferation and differentiation.

Hybrid nanoparticles (HNPs) are described as a combination of both organic and inorganic NPs, in order to obtain multiple and synergistic properties in a single NP, taking advantages of the drug delivery by organic NPs and heat delivery prompted by inorganic NPs, matching the most effective treatment to the individual patient, and overcoming the shortcomings of the current treatments.

Hybridization of effective treatments may enable combination therapy, leading to higher success rates in antitumor efficacy and better quality of life for patients. Multimodality is a progress in cancer therapy due to the higher efficacy of combinational treatment and the synchronized monitoring of therapeutic effects.

HNPs retain the advantages of usNLCs, the NIR-sensitivity of AuNRs, and the active targeting ability of both. They become a robust nanosystem, which comprises three distinct functional components: (i) a lipophilic core where poorly water-soluble drugs can be encapsulated, thereby enhancing drug encapsulation efficiency and controlling drug release, (ii) an inorganic shell with photothermal capability able to increase local temperatures above 39° C. after NIR irradiation, and an external layer, targeted to overexpressed transferrin receptors at the blood-brain barrier, resulting in effective targeting to cross the blood-brain barrier and reach the brain tumor.

These facts are disclosed to illustrate the scientific and technical problem addressed by the present disclosure.

### SUMMARY OF THE INVENTION

The present invention discloses a synergistic and targeted approach treating GB by designing a drug and heat delivery system for targeted therapy comprising a functionalized hybrid metal-lipid nanoparticle, where at least two ligands are coupled to the surface of the nanoparticles and entraps at least one pharmaceutically active ingredient.

This strategy combines a chemical attack owing to the drug inside the nanoparticle (usNLCs), and a physical attack through hyperthermia (AuNRs), to boost the possibilities of therapeutic success. The hybrid metal-lipid nanoparticle can efficiently deliver drug and heat to treat GB.

In a further embodiment, the hybrid metal-lipid nanoparticle is directed to the treatment of brain tumors, particularly glioblastoma.

In a further embodiment, the present invention discloses an organic system wherein the lipid-based nanoplatform is a lipid matrix comprising a lipid content of 15% (w/w).

In a further embodiment, the present invention discloses a lipid-based nanoparticle wherein the usNLCs comprise a pharmaceutically active ingredient.

In a further embodiment, the present invention discloses a drug delivery system wherein the pharmaceutically active ingredient is entrapped or intercalated in the usNLCs.

In a further embodiment, the present invention discloses a drug delivery system wherein the pharmaceutically active ingredient is one selected from the group consisting of non-steroidal anti-inflammatory drugs (NSAIDs).

In a further embodiment, the present invention discloses a drug delivery system wherein the drug acts on inhibition of COX-2 (e.g., celecoxib, CXB). CXB has been investigated for its potential anticancer properties in the field of oncology. Overexpression of COX-2 is chronically found in several stages of carcinogenesis, resulting in higher prostaglandin levels in neoplastic tissue.

In a further embodiment, the present invention discloses a lipid-based nanoplatform further comprising a cationic surfactant, as octadecylamine.

In a further embodiment, the lipid-based nanoplatform discloses a process for obtaining the drug delivery system comprising the steps of high shear homogenization followed by high-pressure homogenization.

In a further embodiment, the present invention discloses an inorganic system wherein the gold nanoparticle comprises an excellent localized surface plasmon resonance (LSPR) with the capacity to convert radiation into heat with high efficiency, as they can produce a hyperthermia effect when excited with an NIR laser (750 to 800 nm).

In a further embodiment, the gold-based nanoplatform discloses a process for obtaining the heat delivery system from the seedless synthesis.

In a further embodiment, the present invention discloses a gold-based nanoplatform further comprising a targeting molecule, such as c(RGDfK).

In a further embodiment, there is a linkage between the gold-based nanoplatform and the target molecule, c(RGDfK). The binding is via functional group such as a guanidine.

In a further embodiment, the present invention relates c(RGDfK) as a linker between lipid-based nanoplatform (usNLCs) and gold-based nanoplatform (AuNRs).

In a further embodiment, the linkage between the organic system and the inorganic system comprises a covalent bonding. The bond is an amide bond formed through reaction of the amine group of the octadecylamine and the carboxyl group of the aspartic acid the c(RGDfK).

In a further embodiment, the present invention discloses an inorganic system wherein the amount of gold metal comprising >200 μg/mL.

In a further embodiment, the present invention discloses a hybrid metal-lipid nanoparticle wherein the mean particle size of the lipid-based nanoplatform is lower than 300 nm, preferably 50 to 100 nm.

In a further embodiment, the present invention discloses a hybrid metal-lipid nanoparticle wherein the nanoplatform has a monodisperse size distribution with a polydispersity index lower than 0.3, preferably from 0.1 to 0.2.

In a further embodiment, the present invention discloses a hybrid metal-lipid nanoparticle wherein the ligand is transferrin. The linkage between the hybrid metal-lipid nanoparticle and transferrin comprises an electrostatic interaction.

In a further embodiment, the present invention discloses a hybrid metal-lipid nanoparticle wherein the target ligand delivery system has high binding selectivity for transferrin receptors.

In a further embodiment, the present invention discloses a hybrid metal-lipid nanoparticle for target a molecule overexpressed in blood-brain barrier and/or tumor cells.

In a further embodiment, the invention relates to injectable composition comprising the hybrid metal-lipid nanoparticle of the present invention.

### General Description

In an embodiment, the proposed invention provides a nanotechnological based platform, which in the context of the present invention means any platform comprising nanotechnology, particularly an HNP with a particle size lower than 100 nm, encapsulating a drug and diffusing heat for cancer therapy. The challenges of this construction obtained from different nanoparticles aim to overcome the limitations of both nanoparticles when used alone and boost their properties when combined. The dual targeting focuses on the two main barriers in GB: the blood-brain and blood-brain tumor barriers. To this end, the presence of transferrin (Tf) enables the functionalization of HNPs to target BBB, the first barrier to overcome, and c(RGDfK) for the tumor barrier, a well-established ligand for αvβ₃/αvβ₅-integrin, overexpressed in GB cells, the second barrier to be overcome.

HNPs mediate the efficient intracellular release of the encapsulated drug, and heat, increasing the concentration of the payload at the target site and reducing agent-associated adverse side-effects. Consequently, the synergistic treatment is more effective than single treatment and promotes an improvement of the safety and efficiency for cancer therapy and/or diagnostic.

In a further embodiment, the present invention is highly versatile, as it is subjected to modifications regarding its composition, entrapped drug, or the attached ligands, depending on its purpose (treatment and/or diagnosis, but not limited, of GB and other cancer types or other diseases, as well as other fields of application). The system is functionalized on the surface with one or more internalizing targeting molecules, enabling specific receptor-mediated endocytosis into tumor cells, including but not limited to GB cells.

In another embodiment, the proposed prototype is constructed by covalent linkage between organic (usNLCs, drug delivery system) and inorganic nanoparticles (AuNRs, photothermal capability). In addition, the surface of the nanoplatform is modified with transferrin (Tf), via electrostatic binding HNPs.

In an embodiment, the present disclosure encompasses a drug and heat delivery system for targeted therapy comprising at least one hybrid nanoparticle (HNP), particularly a metal-lipid nanoparticle, comprising at least one lipid carrier and at least one metal-based nanoparticle, preferably wherein the metal is gold, the HNP comprising at least one nanoplatform, preferably a single nanoplatform, and at least one pharmaceutically active ingredient, wherein at least two ligands are coupled to the surface of the nanoplatform and the pharmaceutically active ingredient is encapsulated in the nanoparticle.

In a further embodiment, it is disclosed a system for use in brain tumor treatment, particularly for glioblastoma.

In a further embodiment, it is disclosed a system for use in chemotherapy.

In a further embodiment, it is disclosed a system wherein the ligands are a blood-brain barrier ligand and a tumor-specific ligand.

In a further embodiment, it is disclosed a system, wherein the lipid carrier is an ultra-small nanostructured lipid carrier (usNLC) comprising a lipid matrix from 5 to 20 (w/w), preferably 15% (w/w).

In a further embodiment, it is disclosed a system, wherein the lipid carrier comprises a liquid lipid fraction and a solid lipid fraction, preferably wherein the solid lipid: liquid lipid fraction ratio is of 25:75. In a particular embodiment, it is disclosed a system wherein the solid lipid fraction comprises mono-, di- and triglyceride esters of fatty acids (C10 to C18), the triester fraction being predominant.

In a further embodiment, it is disclosed a system, wherein the lipid carrier comprises non-ionic surfactants, preferably fat free soybean phospholipids from 0.5 to 2% (w/w), preferably 1% (w/w) and polyoxyl 40 hydrogenated castor oil from 1 to 5% (w/w), preferably 5% (w/w).

In a further embodiment, it is disclosed a system, wherein lipid carrier further comprises a cationic surfactant, preferably wherein the cationic surfactant is octadecylamine.

In a further embodiment, it is disclosed a system wherein the pharmaceutically active ingredient is encapsulated, entrapped or intercalated in the lipid carrier.

In a further embodiment, it is disclosed a system, wherein the metal-based nanoparticle is a gold-based nanoparticle, preferably a gold nanorod (AuNR), comprising a gold amount of at least 200 μg/mL.

In a further embodiment, it is disclosed a system, wherein the mean particle size of the nanoparticle is lower than 200 nm, preferably 50 to 100 nm.

In a further embodiment, it is disclosed a system, wherein the nanoplatform has a monodisperse size distribution, preferably with a polydispersity index lower than 0.2, preferably from 0.1 to 0.2.

In a further embodiment, it is disclosed a system, wherein the ligand is selected from cRGDfK peptide and/or transferrin.

In a further embodiment, it is disclosed a system for targeting a molecule which is overexpressed in blood-brain barrier and/or tumor cells.

In a further embodiment, it is disclosed a method to obtain the system of the present disclosure, comprising the steps of:
providing nanostructured lipid carriers and metal-based nanoparticle, preferably gold nanorods (AuNR) having as target the molecule c(RGDfK);
applying to the lipid carriers and metal-based nanoparticles, both a chemo- and a photothermal treatment, wherein at least two ligands are coupled to the surface of the nanoplatform and encapsulates at least one pharmaceutically active ingredient;
preferably establishing a covalent linkage between the ultra-small nanostructured lipid carriers, preferably a lipid-based nanoparticle, and the metal-based nanoparticle, preferably gold nanorods (AuNR).

In a further embodiment, it is disclosed a parenteral composition comprising the system of the present disclosure.

### BRIEF DESCRIPTION OF THE DRAWINGS

The following figures provide preferred embodiments for illustrating the disclosure and should not be seen as limiting the scope of invention.

FIG. 1. Schematic representation of the synthesis of c(RGDfK)-octadecylamine.

FIG. 2. ¹H NMR spectra of the suspensions in H₂O (30% D₂O) of: (a) ST, pH*4.74; (b) c(RGDfK), 5.0 mmol dm⁻³, pH*3.21; (c) usNLCs^{ST} pH*6.04; (d) usNLCs^{ST}-AuNRs^{c(RGDfK)}, pH*5.32; (e) (usNLCs^{ST}-AuNRs^{c(RGDfK)})^{Tf}, pH*5.41, (f) AuNRs^{c(RGDfK)}, pH*3.79; insets containing expansions from 0 to 4.50 and 6.25 to 9.00 for better visualization of the signals; T=273.15 K.

FIG. 3. ¹³C NMR spectra of the suspensions in H₂O (30% D₂O) of (a) usNLCs^{ST}, pH*4.74; (b) c(RGDfK), 5.0 mmol dm⁻³, pH*3.21; (c) usNLCs^{ST-c(RGDfK)}, pH*6.04; (d) usNLCs^{ST}-AuNRs^{c(RGDfK)}, pH*5.32; (e) (usNLCs^{ST}-AuNRs^{c(RGDfK)})^{Tf}, pH*5.41, (f) AuNRs^{c(RGDfK)}, pH*3.79; insets containing expansions from 15 to 90 and 110 to 190 for better visualization of the signals; T=273.15 K.

FIG. 4. ¹H NMR spectra of the suspensions in H₂O (30% D₂O) of (a) c(RGDfK) peptide, 5.0 mmol dm⁻³, pH*3.21; (b) AuNRs^{c(RGDfK)}, pH*7.18; T=273.15 K.

FIG. 5. TEM image of morphology of A) AuNRs, B) HNPs™ (1) (100 μg/mL) and C) HNPs^{Tf} (2) (200 μg/mL).

FIG. 6. A) Absorbance spectra of AuNRs and AuNRs^{c(RGDfK)} and B) Absorbance spectra of usNLCs^{ST}, HNPs^{Tf} (1) (100 μg/mL) and HNPs^{Tf} (2) (200 μg/mL) nanoparticles under wavelengths from 400 nm to 900 nm.

FIG. 7. Thermo-behavior of HNPs^{Tf} according to the AuNRs amount after NIR laser irradiation (750 nm and light dose of 450 J/cm²).

FIG. 8. In vitro cytotoxic effect, as IC₅₀, of celecoxib-loaded HNPs and celecoxib-loaded HNPs^{Tf} incubated with A) HBMEC cells for 4 hours and 24 hours. B) U87 cells for 24 hours and 72 hours. Data are expressed as mean±SD (n=9)

FIG. 9. A) In vitro cellular uptake of celecoxib-loaded usNLCs, celecoxib-loaded HNPs, and celecoxib-loaded HNPs^{Tf} following 0.5, 1, 2, and 4 hours of incubation with HBMEC cells. Data are expressed as mean±SD (n=9, usNLCs vs. HNPs/HNPs^{Tf}**p<0.01; usNLCs vs. HNPs/target HNPs ****p<0.0001). B) The cellular uptake of fluorescent usNLCs, HNPs, and target HNPs in U87 cells at 1, 2, 4, and 8 hours. Data are expressed as mean±SD (n=9). C) TEM images showing the intracellular location of nanoparticles (AuNRs, HNPs, and HNPs^{Tf}) in U87 cells after 8 hours incubation.

FIG. 10. Pharmacokinetic profiles of celecoxib in solution and encapsulated within nanoparticles (usNLCs and HNPs^{Tf}) in A) plasma, B) brain, C) spleen, D) liver, and E) kidney. Results are presented as average per organ; error bars represent calculated standard deviation (n=3 for each time point).

FIG. 11. In vivo antitumor efficacy of usNLCs evaluated using an orthotopic Swiss nude mouse model. A) Representation of the tumor growth of control (0.9% NaCl and 0.9% NaCl+PTT), free drugs (temozolomide—i.p. and oral administration-, and celecoxib) and nanoparticles (usNLCs, HNPs^{Tf}, HNPs^{Tf}+PTT) B) Kaplan-Meier survival curves of animals administered with free drugs vs. nanoparticles. ****p<0.0001 by Mantel-Cox test. Data presented as mean±SD (3≤n≤6); and C) change in body weight of mice before, during and after the treatments.

FIG. 12. Representative weekly (21, 28, 35, 42, 49, 56, and 91 days) magnetic resonance imaging (MRI) measurements of glioblastoma-bearing mice, analysis a coronal section view.

FIG. 13. Schematic representation of the behaviour of the biomarkers in the present of different drugs and control.

### DETAILED DESCRIPTION

In an embodiment, the present disclosure aims at developing a versatile hybrid metal-lipid nanoparticle, which combines the ability to deliver drugs and to diffuse heat by incorporating therapeutic or diagnostic agents, and covalently attaching inorganic systems, respectively. At the surface, there are specific targeting molecules, particularly to target glioblastoma tumor cells and receptors present in the blood-brain barrier. When administered to a subject, the proposed system allows the delivery of therapeutic agents and heat after light activation, in a controlled release profile, to target cells, particularly glioblastoma tumor cells.

The term “hybrid nanoparticle” or “metal-lipid nanoparticle” or “gold-lipid nanoparticle” refers to a physicochemical complex drug/heat delivery system. This is a single and multimodal system, which seems to be one of the most promising features of NPs application. HNPs are described as a combination of both organic and inorganic NPs, in order to combine multiple, synergistic properties in a single NP, taking advantage of drug delivery by organic NPs and photothermal agents or imaging prompted by inorganic NPs. Examples of “hybrid nanoparticles” include but are not restricted to polymer-hybrid nanoparticles (e.g., polymer-gold nanoparticles, polymer-IONs), lipid-enveloped hybrid nanoparticles (e.g., liposome-gold nanoparticles, liposome-SPIONs), polymeric-gold, micelles hybrid nanoparticles (e.g., metal oxide-peptide amphiphile micelles), among others. Preferably, the metal-lipid nanoparticles are composed of, but not limited to, ultra-small nanostructured lipid carrier and gold nanorods, combining drug delivery and heat delivery, respectively.

The term “encapsulated” refers to the entrapment, or intercalation of the therapeutic agents within the nanoparticle, stressing that the agent or a combination are entrapped in the inner core of the system.

The terms “drug” is employed to designate an organic chemical, a nucleic acid, a peptide, protein, antibody, growth factor or a fragment thereof presenting a linear or cyclic conformation, or a nanostructure expected to significantly alter cell function or the status of cells to which it is delivered to. The drug considered in the proposed nanosystem is celecoxib (CXB). CXB, a non-steroidal anti-inflammatory drug (NSAIDs), inhibits the synthesis of prostaglandins by the inhibition of cyclooxygenase-2 (COX-2) in humans, having demonstrated efficiency to treat inflammatory diseases, such as rheumatoid arthritis and osteoarthritis, ankylosing spondylitis, acute pain, and primary dysmenorrhea. CXB may exert anticancer effects due to their ability to induce apoptosis, inhibit angiogenesis, and enhance cellular immune responses, which are signalling pathways common to both inflammation and carcinogenesis.

The terms “targeting molecule” designate the ligands attached to the nanoparticle that provide advantages in site-specific delivery, reduces the off-site drug cytotoxicity, and increases drug within tumors. The targeting molecule can be, but is not limited to, cell-penetrating peptides, tumor-targeting peptides, proteins, surfactants, or an antibody thereof. The targeting molecules c[RGDfK] and/or transferrin of this invention are used, respectively, as a linker between nanoparticles and to the surface of the metal-lipid nanoparticle. Both peptides are described as molecules with a high affinity to blood-brain barrier or glioblastoma cells, improving the delivery of drugs specifically to the brain.

The term “tumor” is related to abnormal growth of tissue and encompasses all cells present in the tumor microenvironment, including bulk GB cells with limited self-renewal characteristics, GB stem cells with extensive self-renewal and tumorigenic potential, supporting stroma and angiogenic blood vessels that infiltrate the tumor cell mass.

In an embodiment, the system of the disclosure is constructed by covalent linkage between organic (usNLCs, drug delivery system) and inorganic nanoparticles (AuNRs, photothermal capability), including a combination of chemo- and photothermal therapy approaches designed to improve GB treatment. The construction of hybrid lipid-gold nanoparticles relies on celecoxib-loaded lipid nanoparticles (as organic components and drug delivery system) in the core and gold nanorods (as inorganic component and a heat delivery system) in the shell.

In an embodiment, the usNLCs were prepared by the hot high-pressure homogenization (hot HPH) technique. The lipid phase, at 15% w/w and composed of hard fat (Mono-, di-, and triglyceride esters of fatty acids [C10 to C18]), propylene glycol monocaprylate (type I) in a solid: liquid lipid ratio of 25:75, 1% (w/w) of fat free soybean phospholipids (70% phosphatidylcholine), 5% (w/w) of polyoxyl 40 hydrogenated castor oil, 5% (w/w) of celecoxib. Octadecylamine was added to the lipid phase to promote a charge reversion to positive values. The resulting dispersion is then kept stored at 4° C.

In an embodiment, the AuNRs are prepared by seedless synthesis performed for 3 hours at 26° C., yielding a solution with brownish appearance. The particles were purified by centrifugation and re-suspended in an aqueous c(RGDfK) solution (8.28 mM) to increase stability and biocompatibility and improve the targeting ability of the AuNRs. After an incubation period of 2 hours at room temperature, excess peptide was removed from the functionalized AuNRs by centrifugation, twice at 14,000 g for 20 min at 26° C. The as-prepared modified AuNRs^{c(RGDfK)} is then kept stored at 4° C.

In an embodiment, the carboxylic group of c(RGDfK) provided by aspartic acid amino acid is activated by the EDC/NHS method. AuNRs^{c(RGDfK)} (c[RGDfK] at a concentration of 8.28 mM) was dissolved in ultrapurified water followed by the addition of EDC and NHS. The reaction mixture was gently stirred for 2 hours followed by the addition of 10 mL usNLCs^{ST} (3 mM, considering the octadecylamine) and stirred for 3 hours. The reaction mixture was dialyzed against ultrapurified water for 4 hours using a 15 kDa cutoff membrane to remove unreacted reagents.

In an embodiment, targeted HNPs (HNPs^{Tf}) are prepared by electrostatic interaction with a protein, transferrin (0.012 mM), and incubated overnight. The as-prepared modified HNPs^{Tf} are stored at 4° C. until use.

In an embodiment, for HNPs^{Tf}, NMR results confirmed that lipid-gold synthesis occurs through a covalent interaction between the carboxylic group of aspartic acid of c(RGDfK) peptide present in the surface of AuNRs, and the amine group of octadecylamine present in the surface of usNLCs, which enables uniform distribution of inorganic nanoparticles on the outer surface of organic nanoparticles (usNLCs).

In an embodiment, HNPs^{Tf} exhibited small particle size (<100 nm), low polydispersity index (ca. 0.200), high drug loading (5% w/w), and photothermal behavior dependent on AuNRs concentration. The in vitro and in vivo studies revealed that HNPs^{Tf} could safely and specifically increase the permeability of the blood-brain barrier via the receptor transferrin and facilitate the accumulation of nanoparticles in the tumor region in orthotopic tumor-bearing mice.

Furthermore, chemo/photothermal therapy enhanced the therapeutic effect in glioblastoma by delaying tumor growth by 113% when compared with 43% for mice administered with the nanoparticle non-irradiated, and prolonging survival of tumor-bearing mice, with a favorable impact upon the adverse effect profile.

Example 1 below presents the gold-lipid nanoparticle construction. The attachment of the lipid nanoparticles to the gold nanoparticles occurs through the binding of the amine group in octadecylamine and the carboxyl group of aspartate residue in the c(RGDfK) peptide.

Example 2 below presents the parameters used to generally characterize hybrid formulations at the level of their particle size (PS), polydispersity index (PI), zeta potential (ZP), and gold content, as NP critical quality attributes. HNP nanoparticles exhibited small particle size (<100 nm), low polydispersity index (ca. 0.200), high drug loading, and photothermal behavior dependent on AuNRs concentration (FIG. 7). Therefore, NPs should be preferentially small with a monomodal and monodisperse size distribution. ZP is another important parameter that has a direct impact on long-term stability and to sustain the success of the HNPs functionalization.

In an embodiment, the functionalization of the proposed HNPs is performed through the attachment of transferrin targeting ligand onto their surface. The HNPs^{Tf} are designed and prepared aiming at evaluating their potential targeting to blood-brain barrier and GB tumor cells.

c(RGDfK) is a small and stable non-immunogenic water-soluble compound. Considering the overexpression of αvβ₃/αvβ₅-integrin in several cancer cells, including GB, epithelial, ovarian, cervical, breast, lung, kidney and colorectal, and due to the retention of its activity when conjugated with drugs or other molecules, c(RGDfK) presents itself as a potential enhancer of the internalization of anticancer drugs by tumor cells and neovasculature.

In an embodiment, the development of HNPs^{Tf} is an interesting strategy for the treatment of GB, providing the suitable properties to enhance both tumor targeting and anti-tumor activity.

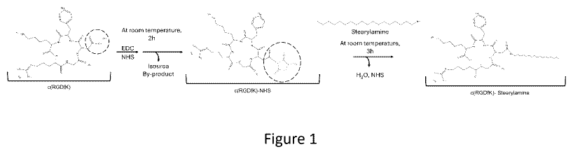

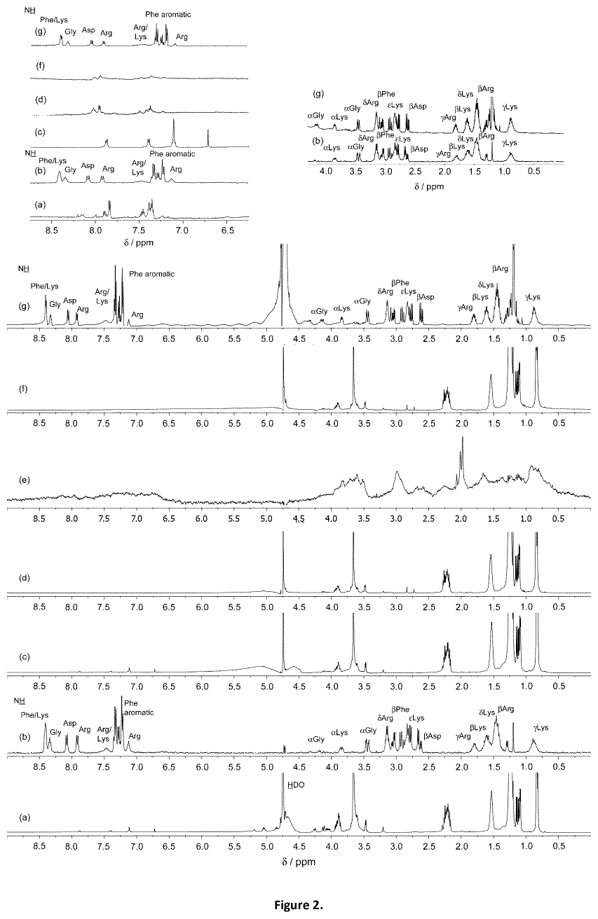

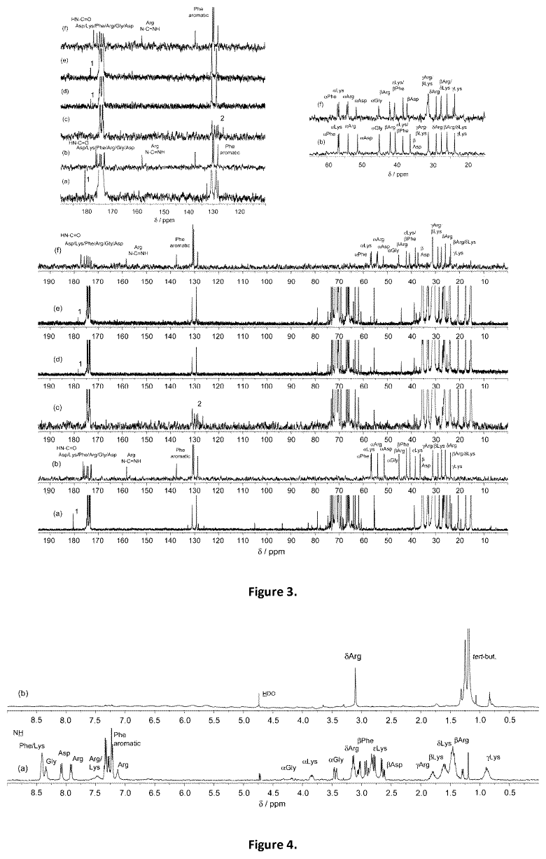

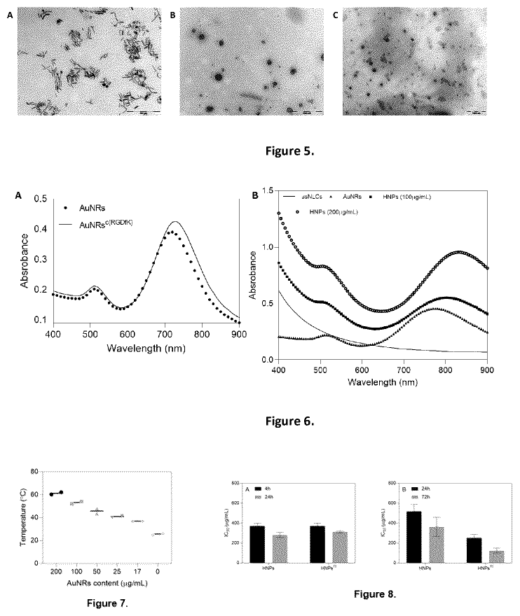

The nature and extent of the interactions of the conjugation between lipid and gold nanoparticles are corroborated by NMR analysis (FIGS. 2, 3 and 4). No significant changes in particle size or polydispersity index following the sequential addition of usNLCs^{ST} and AuNRs^{c(RGDfK)} were observed. The zeta potential increases in usNLCs^{ST} (compared to usNLCs), and the subsequent addition of AuNRs^{c(RGDfK)} leads to a decrease in this property due to the covalent bond between the amine group of octadecylamine and the carboxyl group of aspartate residue present in AuNRs^{c(RGDfK)} (FIG. 1). The obtained HNPs were characterized by UV-vis spectra, which showed that the maximum plasmon peak did not change when both nanoparticles were combined (see FIG. 6A). Different concentrations of AuNRs were also tested (100 μg/mL vs. 200 μg/mL), see Example 3. The peak height at 765 nm for AuNRs increased significantly at high concentrations of gold nanoparticles, while usNLCs did not display a peak in the spectrum around this wavelength range (FIG. 6B). Drug loading was not influenced by the addition of AuNRs to lipid nanoparticles, with a final drug concentration of ~6.9 mg/ml for celecoxib. The ZP of HNPs^{Tf} showed a shift to negative values with the subsequent addition of the transferrin, due to the electrostatic interactions of the carboxyl moieties of c(RGDfK) with the amine groups of octadecylamine (Table 1).

In vitro toxicity studies were performed as described in Example 4. To assess the potential cytotoxicity of both loaded and unloaded NPs, the resazurin assay was conducted in HBMEC and U87 cells. The sensitivity of GB cells to free CXB and TMZ was assessed following a treatment with a drug solution in the range 7-1700 μM and 5-2500 μM, respectively. After 24 hours and 72 hours of incubation, CXB was able to impair cellular proliferation. Celecoxib exhibited lower IC₅₀ values (below 400 M) than temozolomide (800 μM) for all glioma cells.

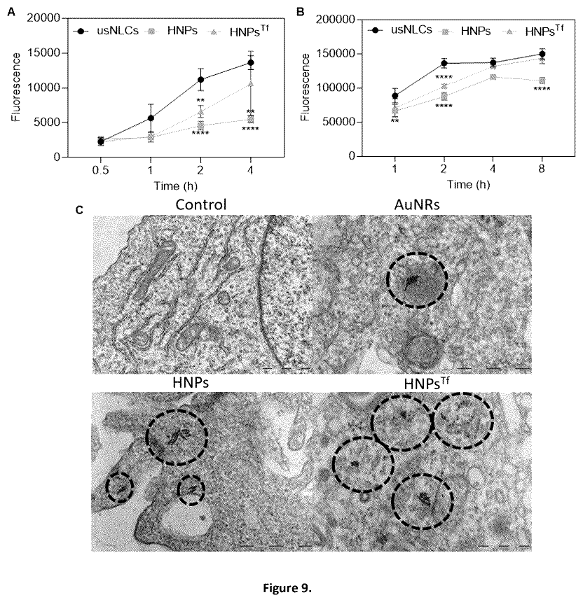

The influence of the surface modification, as well as the potential cytotoxicity of the HNPs were evaluated, indicating a concentration- and time-dependent cytotoxic effect of the HNPs on HBMEC and U87 cells in the concentration range of 2.4-2500 μg/ml (considering the lipid content). In HBMEC cells, the IC₅₀ values of HNPs and HNPs^{Tf} were calculated after 4 hours of incubation yielding 371±23 μg/ml and 377±26 μg/mL, respectively, while after 24 hours of incubation, the IC₅₀ were 280±21 μg/ml and 311±10 μg/mL, respectively (see FIG. 9A). In U87 cells, the IC₅₀ values of the HNPs and HNPs^{Tf} were calculated after 24 hours of incubation and were 516±62 μg/ml and 361±84 μg/ml; while after 72 hours of incubation, the IC₅₀ were 250±32 μg/ml and 124±21 μg/mL, respectively. These results showed a higher cytotoxicity profile of HNPs^{Tf} (see FIG. 9B).

In an embodiment, the addition of both nanoparticles in the same system improved the cytotoxic profile compared to lipid nanoparticles. HNPs (1.39- and 1.20-fold, respectively) and HNPs^{Tf} (1.37- and 1.08-fold, respectively) showed increased cytotoxicity at 4 hours and 24 hours in HBMEC compared to usNLCs. In U87 cells, cytotoxic differences were more pronounced than in usNLCs at 24 hours and 72 hours (HNPs were 1.04- and 1.66-fold, and HNPs^{Tf} were 1.49- and 3.34-fold). These results are consistent with the tumor growth inhibition data, indicating a higher anti-tumor activity of HNPs than of usNLCs.

As expected, surface functionalized HNPs (HNPsTr) induce a lower cell viability, when compared to the other formulations under study. Overall, the results demonstrate that the targeting molecules improved the interaction of HNPs with cells and play an important role in the cytotoxicity profile of the nanosystems. This was further confirmed by permeability studies in HBMEC and cellular uptake studies. The permeability studies showed the benefit of the targeting molecule. The presence of transferrin on the surface of HNPs (Pe=20.6±3.42×10⁵ m/s at 4 hours, see Table 3) increased the BBB permeability. This was because HBMECs expressed Tf receptors enhanced the affinity of HNPs^{Tf} and benefited from receptor-mediated endocytosis. The cellular uptake studies were performed in both cells, HBMEC and U87 cells. Rodhamine-123-loaded HNPs at 200 μg/ml of lipid content were added and incubated for 1, 2, 4, and 8 hours, at 37° C. in 5% CO₂, and analyzed by flow cytometry. Internalization was significantly higher in the U87 cells than in the blood-brain barrier cells (HBMEC), with mean fluorescence 10-fold higher in U87 cells than in HBMEC. These results indicate that the nanoparticles are internalized more readily and rapidly in U87 cells than in HBMEC. FIGS. 9A and B show the effect of surface functionalization in HBMEC and U87 cells. The presence of transferrin favored internalization in HBMEC cells (HNPSTf=1.2±0.1 vs. HNPs=1.0±0.1) at the initial time point. These results are important because the BBB is the first barrier that nanoparticles must overcome to reach the brain tumor. At all the time points, the HNPs^{Tf} values in U87 cells were higher than those of HNPs but lower than those of usNLCs (usNLCs/HNPs^{Tf}<1).

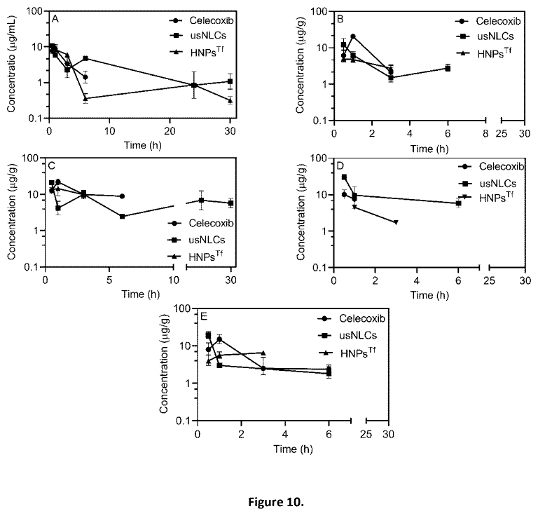

The in vivo studies aimed at characterizing and understanding the pharmacokinetics of CXB after intraperitoneal (i.p.) administration of usNLCs and HNPs formulations, in comparison to a control (free CXB) in the same concentration (Example 5). FIG. 10 and Table 4 show the pharmacokinetic profiles and parameters, respectively. The Cₘₐₓ of i.p. administered celecoxib in solution was 9.09 μg/ml after 1 hour (tₘₐₓ value). In contrast, the encapsulation of celecoxib in usNLCs and HNPs^{Tf} showed an improvement, with a Cₘₐₓ of 10.65 μg/ml and 8.80 μg/mL after 30 min, respectively. The extent of celecoxib absorption from usNLCs, expressed by AUCo-last, was significantly higher for usNLCs (85.4 min×μg/mL) in comparison to HNPs^{Tf} and celecoxib in solution (46.1 min×μg/mL vs. 28.1 min×μg/mL, respectively). Celecoxib encapsulated in nanoparticles reached the brain faster (0.5 hours) than celecoxib in solution (1 hour). These favorable results can be attributed to the small particle size, and lipophilic nature of the nanocarriers, which together improve their membrane permeability. The usNLCs improved the blood-brain barrier crossability of the celecoxib. Note that, however, t_{1/2} and MRT (mean residence time) are higher in plasma and brain when the drug is encapsulated. According to these results, usNLCs seem to have a preferential targeted behavior to the brain and a higher elimination by clearance organs. In turn, only HNPs^{Tf} had a reduced elimination by clearance organs (liver and spleen).

In an embodiment, an orthotopic U87 glioblastoma model in Swiss nude mice was further used to evaluate (i) the efficacy of celecoxib as repurposed anticancer drug, (ii) the benefit of encapsulation of celecoxib in lipid nanoparticles and (iii) the efficacy of a hybrid system, compared with the standard drug, temozolomide.

The safety and tolerability of the free drugs (CXB and TMZ) and formulations (usNLCs and HNPs^{Tf}) were assessed, since the selected treatment regimen required two cycles of five consecutive administrations, separated by two days. The daily intraperitoneal administration of nanoparticles (20 mg/kg) to mice did not elicit significant changes in their body weight (data not shown). However, the celecoxib in solution exhibited toxicity at doses higher than 5 mg/kg. The dose used for temozolomide was 30 mg/kg (i.p administration) and 50 mg/kg (oral administration), as the upper threshold tolerated by animals. The treatment started 21 days after U87 cell implantation.

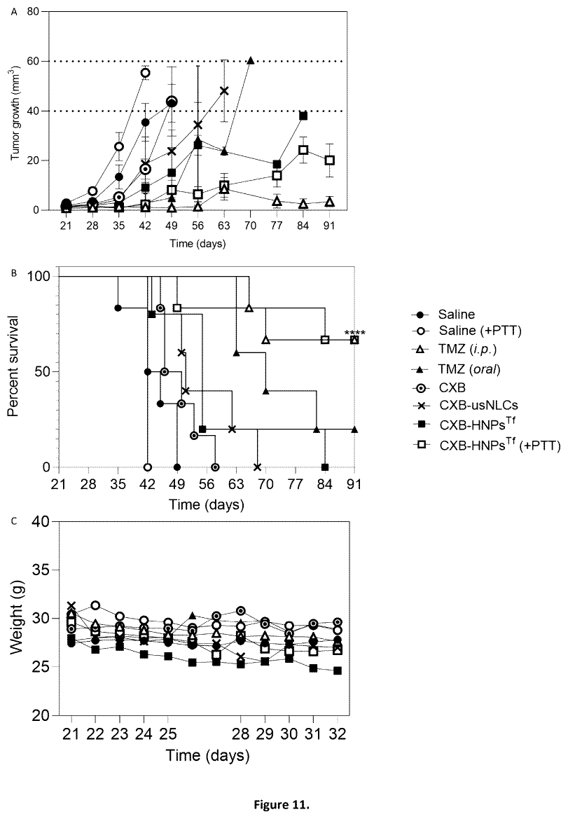

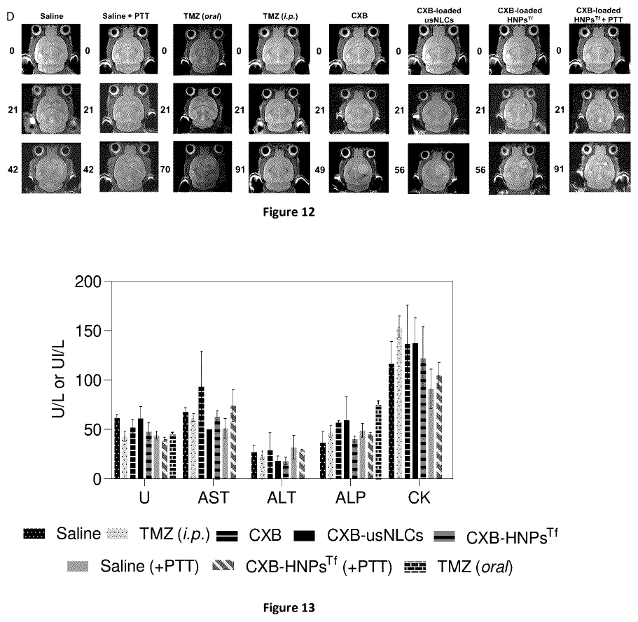

Weekly MR imaging measurements of tumors were conducted in order to evaluate the impact of treatments on tumor size and growth and, consequently, their antitumor efficacy, as presented in Example 5. According to FIG. 11, there were a tumor growth suppressing effect by lipid nanoparticles (celecoxib-loaded usNLCs), hybrid nanoparticles (celecoxib-loaded HNPs^{Tf}), and temozolomide in solution (i.p. and oral administration), compared with saline and celecoxib solution. Tumor growth was more pronounced in the control group during the 21^{st} and 34^{th} days, which corresponds to the treatment period. Celecoxib solution and celecoxib-loaded usNLCs inhibited the growth of GB cells, but the effect was not significant; and the mice were mainly sacrificed at 48 and 51 days after the tumor implantation, see FIG. 11B. The control group behavior and free celecoxib were similar, meaning that the amount of free celecoxib was not sufficient to treat the tumors. In addition, temozolomide solution and HNPs^{Tf} groups exhibited a therapeutic effect on glioblastoma tumors. The therapeutic performance of celecoxib-loaded HNPs^{Tf} showed a significant result in 1 animal that survived to day 84 after tumor implantation (FIG. 11 B). Despite this effect, these results suggest that celecoxib-loaded HNPs^{Tf} may not be able to prevent the tumor progression. Temozolomide (i.p. administration) in solution was the only successful treatment at the end of 91 days after tumor implantation. However, out of 6 animals studied, only 4 survived and prevented tumor recurrence. But, when the temozolomide was administered orally, the results were not so favorable, in which only one animal survived, without tumor recurrence (MST=70 days). The mean survival time (MST) of the HNPs^{Tf} was 55 days, indicating a better anti-glioblastoma effect compared to the control group (43 days), followed by celecoxib in solution (48 days) and celecoxib-loaded usNLCs (51 days). These results indicate that HNPs^{Tf} exhibited a higher anti-glioblastoma effect, as substantiated in cellular studies (Example 4). The mean survival time (MST) of nanoparticle groups improved compared to the control groups and celecoxib in solution. Throughout the treatment process, the weight of the mice remained stable, with some changes during the 12 days of administration, but all animals regained weight by the end of the study (FIG. 11C)

In an embodiment, to further confirm the phototherapeutic effects of HNPs^{Tf} in terms of antitumor efficacy, a second in vivo efficacy study was performed (Example 5) to demonstrate that the HNPs^{Tf} can be used as a potential nanoparticle for chemo/photothermal therapy in vivo. The treatment started 21 days after U87 cell implantation. To ensure an enough amount of AuNRs in brain at irradiation time and, consequently, the photothermal effect, more than 200 μl of unloaded HNPs^{Tf} (placebo) were administrated. This strategy ensures the photothermal effect since the Au concentrations in the brain exceed 20 μg/mL. The treatment regimen was the same as mentioned above (5 consecutive days, 2 days off, and another 5 consecutive days), and the animals were irradiated every 6 hours after administration. Tumors in the saline (+PTT) grew up to 40 mm³ within 42 days (20-fold in volume since the start of treatment). This result shows that the laser has no effect in tumor growth. Tumor volume in celecoxib-loaded HNPs^{Tf} (+PTT) irradiation group grew to 10 mm³, which is 10 times the volume since the start of laser treatment. Celecoxib-loaded HNPs^{Tf} (+PTT) slowed tumor growth with a 4.10-fold inhibition of tumor volume from day 21 (first day of treatment), which is considered statistically different compared with the saline group. Importantly, these results indicate that the single treatment with celecoxib-loaded HNPs^{Tf} was not sufficient to eliminate the tumors; however, the group treated with celecoxib-loaded HNPSTf+PTT exhibited an efficient tumor growth inhibition during the 91 days post-injection (FIGS. 11 and 12). To understand the impact of different treatments, tumor growth delay (TGD) and tumor growth inhibition (TGI) were used to assess the effects of treatments on tumor progression (Table 2). The MST of the celecoxib-loaded HNPs^{Tf}+PTT group was 91 days, and the antitumor effect was remarkably higher (98% growth delay and 78% of inhibition growth compared to the corresponding saline group), see Table 5. The tumors treated with celecoxib-loaded HNPs^{Tf}+PTT exhibited a higher percentage of TGD compared to temozolomide (i.p. and oral administration), with growth delays of 85%, 75%, and 54%, respectively. Concerning TGI, the results demonstrate the effect of encapsulation of CXB, which allowed administration of a higher dose (5 mg/kg CXB in solution vs. 20 mg/kg CXB encapsulated) and confirms that tumor size did not increase during the administration period (FIG. 10). Also, laser-irradiated HNPs^{Tf} prompted a tumor inhibition efficiency that was 1.18 and 1.86 times higher than temozolomide (i.p. and oral administration, respectively), with a TGI of 78% compared to 66% and 42%. This result provides a proof of concept that celecoxib-loaded HNPs^{Tf} can accumulate and effectively release celecoxib into the brain by NIR irradiation, leading to effective inhibition of tumor growth (FIG. 11).

In a surprising way, the effects of chemo- and photothermal therapy synergistically contributed to the anti-glioblastoma effect of HNPs^{Tf}, which is attributed to brain accumulation of the drugs and glioblastoma targeting. Chemo- and photothermal therapy reduced the local recurrence and demonstrated the ability to prolong the survival rate in this animal model.

Furthermore, daily injections of NPs showed a better safety profile than the administration of non-encapsulated drugs (CXB and TMZ). All biomarkers (aspartate aminotransferase (AST), alanine aminotransferase (ALT), alkaline phosphatase (ALP), gamma glutamyl transpeptidase (GGT), creatinine kinase (CK), and urea (U)) showed no statistical differences amongst the saline solution, nanoparticles, and nanoparticles plus irradiation groups. However, a significant increase in CK and AST levels was denoted following the administration of the drugs in solution. CK is an indicator of cardiotoxicity, and CK values were 1.32-fold (temozolomide) and 1.17-fold (celecoxib) higher than the saline group after the treatment with free drugs, whereas no increase was observed after treatment with nanoparticles. Cardiotoxicity is a known side effect of temozolomide. Additionally, the biomarker values for mice administrated with HNPs^{Tf} (with or without NIR irradiation) were similar to saline groups (with or without NIR irradiation). No significant variations on the levels of serum GGT were detected (Table 5).

These outcomes suggest that the celecoxib-loaded HNPs^{Tf} exhibits a synergistic chemo/photothermal effect and biocompatibility, thus warranting further in vivo studies and potential clinical translation to treat glioblastoma patients.

### EXAMPLES

The following set of examples is intended to illustrate but not limit the present invention.

### Example 1—Gold-Lipid Construction

AuNRs^{c(RGDfK)} modification was achieved by amine coupling (FIG. 1). The carboxylic group of c(RGDfK) provided by aspartic acid amino acid was activated by the EDC/NHS method. c(RGDfK) (8.28 mM) was dissolved in ultrapurified water followed by the addition of EDC (1.6×10⁻⁵ mmol) and NHS (1.6×10⁻⁵ mmol). The reaction mixture was gently stirred for 2 hours followed by the addition of 10 ml usNLCs^{ST} (3 mM, considering the octadecylamine) and stirred for 3 hours. The reaction mixture was dialyzed against ultrapurified water for 4 hours using a 15 kDa cutoff membrane to remove unreacted reagents. Targeted HNPs (HNPs^{Tf}) were prepared by electrostatic interaction with a protein receptor molecule, transferrin (0.012 mM), and incubated overnight. The as-prepared modified targeted hybrid nanoparticles (FIG. 7.2) were stored at 4° C. until use.

### Example 2—Physicochemical Characterization

The mean PS and PI were measured by dynamic light scattering (DLS) using a Zetasizer Nano ZS (Malvern Instruments, Malvern, UK) set at a detection angle of 173° at 25° C. The size of a particle is calculated from the translational diffusion coefficient by using the Stokes-Einstein equation. Cumulants method was used for data analysis. ZP was determined in the same apparatus, set at a temperature of 25° C., according to the Helmholtz-Smoluchowsky equation. Nanodispersions were suitably diluted in ultra-purified water and analyzed three times. The results are represented as mean±standard deviation. The obtained HNPs were characterized by UV-vis spectra, which showed that the maximum plasmon peak did not change when both nanoparticles were combined. Different concentrations of AuNRs were also tested (100 μg/mL vs. 200 μg/mL), and no changes in particle size or polydisperse index were observed. The peak height at 765 nm for AuNRs increased significantly at high concentrations of gold nanoparticles, while usNLCs did not display a peak in the spectrum around this wavelength range. Drug loading was not influenced by the addition of AuNRs to lipid nanoparticles, with a final drug concentration of ~6.9 mg/ml for celecoxib.

TABLE 1
Physicochemical characteristics, particle size (PS), polydispersity index
(PdI), and zeta potential (ZP) of freshly purified formulations.

| | PS | | ZP | Au content | DL |
|---|---|---|---|---|---|
| | (nm) | PdI | (mV) | (μg/mL) | (%) |
| usNLCs | 59.9 ± 0.3 | 0.152 | −37 ± 3 | — | 4.5 ± 0.1 |
| usNLCs^{ST} | 64.1 ± 0.2 | 0.184 | 39 ± 2 | — | 4.2 ± 0.1 |
| AuNRs^{CTAB} | — | — | 38 ± 2 | 37 | — |
| AuNRs^{c(RGDfK)} | — | — | −1 ± 1 | 32 | — |
| HNPs | 62.6 ± 1.2 | 0.134 | 32 ± 1 | 200 | 4.6 ± 0.6 |
| HNPs^{Tf} | 60.4 ± 0.1 | 0.115 | 29 ± 2 | 200 | 4.5 ± 0.3 |

Data are expressed as mean ± SD (n = 3, usNLCs vs. other nanoparticles **** p < 0.0001).
Key: ST—octadecylamine; c(RGDfK)—cyclo-(arginine(R)-glycine(G)-aspartic acid(D)-phenylalanine-lysine); Tf—Transferrin; HNPs—Hybrid Nanoparticles; AuNRs—Gold Nanorods; N.A.—not applicable; Au—gold; DL—drug loading.

### Example 3—Photothermal Conversion

AuNRs and HNPs/HNPs^{Tf} displayed unambiguous photothermal therapy by localized surface plasmon resonance (LSPR), responding to the 765 nm Near-infrared radiation (NIR) wavelength. FIG. 6 present the changes in the temperature profile of HNPs/HNPs^{Tf} increasing up to 60° C. The highest temperature (at 200 μg/ml) obtained was near the temperature of naive AuNRs.

### Example 4—In Vitro Toxicity Studies

The cytotoxicity effect of AuNRs^{c(RGDfK)} and HNPs/HNPs^{Tf} against HBMEC. Regarding cell viability, HBMEC were cultured EndroGRO™ medium, supplemented with rh-EGF, L-Glutamine, hydrocortisone hemisuccinate, heparin sulfate, ascorbic acid and FBS, 1% (v/v) penicillin-streptomycin solution. HBMEC were maintained at 37° C. in a humidified atmosphere containing CO₂ (5%). The resazurin assay was used to determine the cytotoxicity of CXB, AuNRs^{c(RGDfK)} and HNPs/HNPs^{Tf}. Briefly, 2000 cells per well were seeded in a 96 well plate and incubated for 4 hours and 24 hours after the medium was replaced with increasing concentrations of CXB or AuNRs^{c(RGDfK)} or HNPs/HNPs^{Tf}. Subsequently, the medium was removed, and the cell viability determined. For this purpose, 100 μl of 10% (w/v) resazurin solution in EndroGRO™ was added to the cells and incubated for approximately 2 hours, at 37° C. The enzymatic reduction of resazurin to resorufin was analyzed by UV/Vis spectrophotometry at 570 nm and 600 nm. Cell viability was assessed indirectly according to equation 1.

$$\% Cell Viability = 100 \times \frac{{( {Abs}_{570 nm} - {Abs}_{600 nm} )}_{Treated Cells}}{{( {Abs}_{570 nm} - {Abs}_{600 nm} )}_{Control}} \qquad ( eq . 1 )$$

The cytotoxicity effect of AuNRs^{c(RGDfK)} and HNPs/HNPs^{Tf} against U87 cells. Regarding cell viability, the U87 cells were cultured in DMEM/F-12 medium, supplemented with 10% (v/v) FBS, 1% (v/v) penicillin-streptomycin solution and sodium bicarbonate. U87 were maintained at 37° C. in a humidified atmosphere containing CO₂ (5%). The resazurin assay was used to determine the cytotoxicity of CXB, AuNRs^{c(RGDfK)} and HNPs. Briefly, 2000 cells per well were seeded in a 96 well plate and incubated for 24 hours or 72 hours after the medium was replaced with increasing concentrations of CXB or AuNRs^{c(RGDfK)} or HNPs/HNPS^{Tf}. Subsequently, the medium was removed, and the cell viability determined. For this purpose, 100 μl of 10% (w/v) resazurin solution in DMEM/F 12 was added to the cells and incubated for approximately 2 hours, at 37° C. The enzymatic reduction of resazurin to resorufin was analyzed by UV/Vis spectrophotometry at 570 nm and 600 nm. Cell viability was assessed as described above.

All experiments were performed in triplicate and the total lipid content of the particles reflecting a 50% reduction in cell viability (IC₅₀) was determined from the concentration-response curves (see FIGS. 7A and B).

Permeability studies were performed in a BBB model using Transwell® devices in which cells were grown for 7 days until monolayer formation, and its integrity was assessed by the transendothelial electrical resistance (TEER) and Lucifer yellow permeability (LY, used at the end of the experiment). The presence of transferrin on the surface of HNPs (Pe=20.6±3.42×10⁻⁵ cm/s at 4 hours) increased the BBB permeability. This was because HBMECs expressed Tf receptors enhanced the affinity of target HNPs and benefited from receptor-mediated endocytosis. The effect of HNPs on BBB permeability was in the order HNPs^{Tf}>HNPs>usNLCs.

TABLE 3
Apparent permeability coefficient (Pₑ) values
and ratio of CXB-loaded nanoparticles.

| | Pₑ | |
|---|---|---|
| NPs | (cm/s) × 10⁻⁵ | Ratio usNLCs |
| usNLCs | 7.4 ± 0.1 | 1 |
| HNPs | 12.1 ± 0.7 | 1.9 ± 0.1 |
| Target HNPs | 21 ± 3**** | 3.3 ± 0.1*** |

Data are expressed as mean ± SD (n = 3, usNLCs vs. HNPs^{Tf}, ****p < 0.0001).

Concerning cell uptake assay, HNPs cellular uptake by HBMECs was further studied by flow cytometry (ACEA NovoCyte®, ACEA Biosciences, Inc., USA). For that, 0.5 mL of cell suspension with 100 000 cells per well were seeded 24 hours before the experiments into 24 well tissue culture test plates. The samples of Rhodamine-123-loaded HNPs at 200 μg/ml of lipid content were added and incubated for 0.5 hours, 1 hour, 2 hours, and 4 hours at 37° C. in 5% CO₂. Free Rhodamine-123 was removed by ultrafiltration using centrifugal filter devices (Amicon® Ultra, Ultra cell-50k, Millipore, USA), thereby removing its interference in the measurements. The control group was treated following the same steps without the presence of nanoparticles. After incubation, cells were washed three times with cold PBS to remove free HNPs and trypsinized. Fluorescence of Rhodamine-123 (Aexc=488 nm, em=530 nm) was measured. Then, cells were centrifuged at 1500×g for 5 min, resuspended in PBS and analyzed through flow cytometry. Cell uptake of HNPs by HBMEC is displayed in FIG. 8A.

Concerning cell uptake assay, HNPs cellular uptake by U87 MG cells were further studied by flow cytometry (ACEA NovoCyte®, ACEA Biosciences, Inc., USA). For that, 0.5 ml of cell suspension with 100 000 cells per well were seeded 24 hours before the experiments into 24 well tissue culture test plates. The samples of Rhodamine-123-loaded HNPs at 200 μg/ml of solid content were added and incubated for 1 hours, 2 hours, 4 hours and 8 hours, at 37° C. in 5% CO₂. Free Rhodamine-123 was removed by ultrafiltration using centrifugal filter devices (Amicon® Ultra, Ultra cell-50k, Millipore, USA), thereby removing its interference in the measurements. The control group was treated following the same steps without the presence of HNPs. After incubation, cells were washed three times with cold PBS to remove free NLCs and trypsinized. Fluorescence of Rhodamine-123 (Aexc=488 nm, Xem=530 nm) was measured. Then, cells were centrifuged at 1500×g for 5 min, washed with PBS, centrifuged, resuspended in PBS and analyzed through flow cytometry. Cell uptake of HNPs by U87 MG cells is displayed in FIG. 9B. Cellular TEM images showed that a large amount of target HNPs was internalized in the cytoplasm of U87 cells (see FIG. 9C). These data corroborated the previous cellular uptake data.

### Example 5—In Vivo Biodistribution, Efficacy and Biochemical Analysis

Adult male and female Swiss Nu/Nu and Swiss Nu/+ mice, aged between 10-14 weeks and weighting 22-30 g, were purchased from Charles River Laboratories (Lyon, France) and maintained in local animal facilities under controlled conditions (12 hours light/dark cycle, at 20±2° C. and 50±5% of relative humidity), with free access to standard diet and water. All animal experiments were conducted in agreement with the international regulations of the European Union.

usNLCs and HNPs were administered to mice by intraperitoneal route, in a single dose (20 mg/Kg). A solution containing CXB in PEG 400: NaCl 0.9% (pH=7.4) (2:1, V/V) was used as control. Thereafter, mice were sacrificed by decapitation at predetermined post-dosing time points. Blood samples were collected to heparinised tubes and centrifuged at 2880 g for 10 min at 4° C. Plasma supernatant was collected and frozen at −80° C. until analysis. After exsanguination, the brain, liver, and spleen were carefully excised and immediately weighted. Brain and liver tissues were homogenized with an H₂O-acetonitrile (1:1, V/V) solution (4 mL per g of tissue) using a Thomas® Teflon pestle tissue homogenizer and centrifuged at 4 150 g for 15 min at 4° C. In parallel, spleen tissues were homogenized with 1 ml of the same solution using a high-speed stirrer (Ultra-Turrax X1020, Ystral GmbH, Dottingen, Germany) and then, centrifuged at 12 045 g for 5 min at 4° C. All supernatants were collected and stored at −80° C. until analysis using a previously validated HPLC method. The maximum peak concentration (Cₘₐₓ) in plasma, brain, liver, and spleen of CXB and the corresponding time to reach Cₘₐₓ (tₘₐₓ) were directly retrieved from the experimental data obtained. Taking into account the non-compartmental model and based on the mean concentration values for each time point, the remaining pharmacokinetic parameters were estimated using PKSolver, a freely available menu-driven add-in program for Microsoft Excel written in Visual Basic Applications (VBA) The pharmacokinetic parameters regarding the administration of the CXB, usNLCs and HNPs to mice are displayed in Table 4 and FIG. 9.

TABLE 4
Pharmacokinetic parameters obtained for co-encapsulated CXB in usNLCs and target HNPs, in comparison
to free CXB in solution, in plasma (P), brain (B), liver (L), spleen (S) and kidney (K).

| | CXB | | | | | usNLCs | | | | | Target HNPs | | | | |
|---|---|---|---|---|---|---|---|---|---|---|---|---|---|---|---|
| Organ | P | B | L | S | K | P | B | L | S | K | P | B | L | S | K |
| t_{1/2} (h) | 1.9 | 1.4 | 2.8 | 4.07 | 2.0 | 10.9 | 9.7 | 2.9 | 8.5 | 2.2 | 7.5 | 2.8 | — | — | — |
| tₘₐₓ (h) | 1 | 1 | 0.5 | 1 | 2.0 | 0.5 | 0.5 | 0.5 | 0.5 | 0.5 | 0.5 | 0.5 | 1 | 1 | 3 |
| Cₘₐₓ (μg/mL | 9.1 | 20.6 | 10.6 | 22.4 | 15.6 | 10.7 | 12.8 | 30.6 | 21.1 | 19.8 | 8.8 | 6.0 | 4.6 | 15.6 | 6.6 |
| or μg/g) | | | | | | | | | | | | | | | |
| AUC₀₋ₗₐₛₜ | 28.1 | 36.7 | 27.7 | 73.2 | 35.3 | 85.4 | 64.3 | 42.3 | 178.7 | 18.6 | 46.1 | 11.4 | 8.1 | 36.4 | 26.3 |
| (h × μg/mL | | | | | | | | | | | | | | | |
| or h × μg/g) | | | | | | | | | | | | | | | |
| AUC_{inf} | 32.3 | 39.5 | 39.8 | 126.2 | 42.2 | 103.8 | 70.4 | 67.5 | 918.6 | 24.1 | 49.6 | 22.8 | — | — | — |
| (h × μg/mL | | | | | | | | | | | | | | | |
| or h × μg/g) | | | | | | | | | | | | | | | |
| AUCₑₓₜᵣₐₚ(%) | 13.0 | 7.08 | 30.4 | 42.0 | 16.3 | 17.7 | 8.7 | 37.3 | 80.5 | 22.8 | 7.07 | 50 | | | |
| MRT (h) | 2.9 | 2.0 | 4.6 | 6.47 | 3.05 | 15.3 | 11.9 | 5.1 | 126.9 | 3.7 | 10.4 | 4.23 | — | — | — |

tₘₐₓ—time to achieve the maximum concentration; Cₘₐₓ—Maximum concentration; AUC_{0-inf}—area under the concentration time-curve from time zero to infinite; AUC₀₋ₗₐₛₜ—Area under the concentration time-curve from time zero to the last measurable drug concentration.

Regarding orthotopic U87 human GB tumor model, before the surgical procedure, fourteen nude mice were weighted, given buprenorphine (0.1 mg/Kg) intraperitoneally and placed on a stereotactic frame. To maintain anesthesia and sedation, the mice were anesthetized for 30 min with 4% isoflurane gas, mixed with medical oxygen at a rate of 1 L/min. Vital signs such as respiratory frequency and body temperature were monitored continuously. After the identification of the injection site, at the Cartesian coordinates (x, y, z)=(2.1, 0.5, −3) in relation to bregma, approximately 36 000 U87 cells were injected in the striatum of the frontal cortex of the right hemisphere of the brain, using a Hamilton syringe. In every mouse, the presence, location and volume of the tumors were assessed by Magnetic Resonance Imaging (MRI) weekly. MRI was performed using a 9.4T Bruker BioSpec 94/20USR system (Bruker, Germany) after anesthetizing mice with isoflurane (1 L/min, induction: 4% isoflurane; maintenance: 2% isoflurane). Vital signs, in particular, respiratory frequency and body temperature, were monitored continuously. For structural analysis, T2-weighted images were acquired in sagittal, coronal and axial planes, using a rapid acquisition with relaxation enhancement sequence (T2 Turbo RARE). Tumor images were manually segmented in the 3D Slicer software for 3D reconstruction, in order to calculate tumor volume.

To understand the drug effect: At 21 days post-tumor cell injection, mice were randomly divided into six groups and treated intraperitoneally for two cycles of five consecutive days. The following groups were tested: group 0 (G0), saline solution (n=6); group 1 (G1), free temozolomide, at a concentration of 30 mg/kg (i.p. administrated, animals n=6); group 2 (G2), free temozolomide, at a concentration of 50 mg/kg (oral administrated, animals n=6); group 3 (G3), free celecoxib, at a concentration of 5 mg/kg (animals, n=5); group 4 (G4), celecoxib-loaded usNLCs, at a concentration of 20 mg/kg (animals, n=6); group 5 (G5), celecoxib-loaded HNPs^{Tf}, at a concentration of 20 mg/kg (animals, n=5). Similarly, to the safety evaluation test, all animals were closely observed during the immediate post injection period. Mice physical appearance (body weight and coat condition), body function (food intake), presence of loose stools, diarrhea or blood in diarrhea and behavior (handling, aggression, abnormal gait and posture and reluctance to move) were also monitored throughout the study. Pre-determined human endpoints for animal euthanasia were also established, specifically, a weight loss greater than 20% of initial body weight, a temperature drop (<35° C.), and a tumor size higher than 50 mm³.

To understand the photothermal effect: Mice were randomly divided into 2 groups after 21 days of orthotopic U87 cells engraftment: one group was treated with saline plus NIR irradiation (animals, n=3) and another group with celecoxib-loaded-HNPs^{Tf} plus NIR irradiation, at a concentration of 20 mg/kg (animals, n=6). The day of intraperitoneal administration was designated as day 0. Each group was administered i.p. for 5 consecutive days, paused for two days, and administered i.p. for an additional 5 consecutive days, with laser irradiation occurring 6 hours and 30 hours post administration, which corresponds to maximal brain HNPs concentration. Tumors were exposed to NIR laser irradiation with a light dose of 450 J/cm² (for approximately 12 min).

The tumor growth delayed (TGD), which concerns the evaluation of tumor treatment modalities, was calculated according to

$$TGD = \frac{{D}_{treatment}}{{D}_{control}} \times 100 \qquad ( eq . 3 )$$

where, Dₜᵣₑₐₜₘₑₙₜ and D_{control} correspond to the average doubling times of the treated and control tumors, respectively.

The tumor growth inhibition (TGI), which measures the tumor size between treated and control groups at specific time points was calculated according to

$$TGI = 100 - ( \frac{Δ T}{Δ C} \times 100 ) ( \% )$$

where ΔT (=T−T₀) represents the change in tumor size (volume) for the treated animals, while ΔC(=C−C₀) represents the change in tumor size (volume) for the control animals. By comparing these values, the degree of tumor growth inhibition can be determined.

TABLE 5
In vivo outcomes include the median survival time, survivals at certain time
points, tumor-growth delay, percentage of tumor volume growth and percentage
of tumor volume growth inhibition (data represented as mean ± SD, 3 ≤ n ≤ 6).

| | Median | Survivals | Survivals | % Tumor | % Tumor |
|---|---|---|---|---|---|
| | survival | at 56 | at 91 | Growth | Growth |
| Treatment group | (days) | days | days | Delay | Inhibition |
| Saline* | 43 | 0/6 | 0/6 | N.A. | N.A. |
| Saline + PTT** | 42 | 0/6 | 0/6 | N.A. | N.A. |
| TMZ (oral) | 70 | 1/6 | 1/6 | 54 ± 11 | 42 ± 14 |
| TMZ (i.p.) | 91 | 6/6 | 4/6 | 75 ± 21 | 66 ± 33 |
| CXB | 48 | 1/6 | 0/6 | 15 ± 5 | 13 ± 6 |
| CXB-usNLCs | 51 | 1/6 | 0/6 | 29 ± 7 | 15 ± 6 |
| CXB-HNPs^{Tf} | 55 | 5/6 | 0/6 | 38 ± 12 | 21 ± 8 |
| CXB-HNPs^{Tf} + PTT | 91 | 5/6 | 4/6 | 85 ± 10* | 71 ± 13* |
| | | | | 103 ± 10** | 78 ± 10** |

N.A.—not applicable.

Tumor diameter and volume measured by MRI are introduced in FIG. 11.

The eight groups of glioblastoma-bearing Swiss nude mice considered in the anti-tumor efficacy study, and subjected to two cycles of five consecutive days (separated by two days) for 12 days, were also monitored for toxicity evaluation: (1) control (0.9% NaCl, physiological saline); (2) temozolomide in solution (i.p. administration); (3) temozolomide in solution (oral administration); (4) celecoxib in solution; (5) celecoxib-loaded usNLCs; (6) celecoxib-loaded HNPs^{Tf}; (7) control (0.9% NaCl, physiological saline) plus NIR irradiation; (8) celecoxib-loaded HNPs^{Tf} plus NIR irradiation (+PTT). After reaching the endpoint, animals were euthanized. Whole blood samples were collected from mice immediately to heparinized tubes. The supernatant (serum) of blood obtained after centrifugation (2880×g for 10 min at 4° C.) was used to determine the levels of physiological biomarkers such as aspartate aminotransferase (AST), alanine aminotransferase (ALT), alkaline phosphatase (ALP), gamma glutamyl transpeptidase (GGT), creatinine kinase (CK), and urea (U) to investigate the effects of drug, nanoparticles and photothermal treatment.

The disclosure should not be seen in any way restricted to the embodiments described and a person skilled in the art will foresee many possibilities to modifications thereof.

The above-described embodiments are combinable.

1. Drug and heat delivery system for targeted therapy comprising at least one hybrid nanoparticle (HNP), particularly a metal-lipid nanoparticle, comprising at least one lipid carrier and at least one metal-based nanoparticle, preferably wherein the metal is gold, the HNP comprising at least one nanoplatform, preferably a single nanoplatform, and at least one pharmaceutically active ingredient, wherein at least two ligands are coupled to the surface of the nanoplatform and the pharmaceutically active ingredient is encapsulated in the nanoparticle.

2. System according to the previous claim for use in brain tumor treatment, particularly for glioblastoma.

3. System according to claim 1 for use in chemotherapy.

4. System according to claim 1 wherein the ligands are a blood-brain barrier ligand and a tumor-specific ligand.

5. System according to claim 1, wherein the lipid carrier is an ultra-small nanostructured lipid carrier (usNLC) comprising a lipid matrix from 5 to 20 (w/w), preferably 15% (w/w).

6. System according to claim 1, wherein the lipid carrier comprises a liquid lipid fraction and a solid lipid fraction, preferably wherein the solid lipid: liquid lipid fraction ratio is of 25:75.

7. System according to claim 6 wherein the solid lipid fraction comprises mono-, di- and triglyceride esters of fatty acids (C10 to C18), the triester fraction being predominant.

8. System according to claim 1, wherein the lipid carrier comprises non-ionic surfactants, preferably fat free soybean phospholipids from 0.5 to 2% (w/w), preferably 1% (w/w) and polyoxyl 40 hydrogenated castor oil from 1 to 5% (w/w), preferably 5% (w/w).

9. System according to claim 1, wherein lipid carrier further comprises a cationic surfactant, preferably wherein the cationic surfactant is octadecylamine.

10. System according to claim 1 wherein the pharmaceutically active ingredient is encapsulated, entrapped or intercalated in the lipid carrier.

11. System according to claim 1, wherein the metal-based nanoparticle is a gold-based nanoparticle, preferably a gold nanorod (AuNR), comprising a gold amount of at least 200 μg/mL.

12. System according to claim 1, wherein the mean particle size of the nanoparticle is lower than 200 nm, preferably 50 to 100 nm.

13. System according to claim 1, wherein the nanoplatform has a monodisperse size distribution, preferably with a polydispersity index lower than 0.2, preferably from 0.1 to 0.2.

14. System according to claim 1, wherein the ligand is selected from cRGDfK peptide and/or transferrin.

15. System according to claim 1 for targeting a molecule which is overexpressed in blood-brain barrier and/or tumor cells.

16. Method to obtain the system of claim 1, comprising the steps of:
providing nanostructured lipid carriers and metal-based nanoparticle, preferably gold nanorods (AuNR) having as target the molecule c(RGDfK);
applying to the lipid carriers and metal-based nanoparticles, both a chemo- and a photothermal treatment, wherein at least two ligands are coupled to the surface of the nanoplatform and encapsulates at least one pharmaceutically active ingredient; preferably establishing a covalent linkage between the ultra-small nanostructured lipid carriers, preferably a lipid-based nanoparticle, and the metal-based nanoparticle, preferably gold nanorods (AuNR).

17. Parenteral composition comprising the system of claim 1.
